(19) United States
(12) Patent Application Publication
(10) Pub. No.: US 2017/0151244 A1
(43) Pub. Date: Jun. 1, 2017

(54) ORAL PHARMACEUTICAL COMPOSITION OF TOFACITINIB

(71) Applicant: Sun Pharmaceutical Industries Limited, Mumbai, Maharashtra (IN)

(72) Inventors: Sumit SAHA, Purnea (IN); Swati AGGRAWAL, New Delhi (IN); Mukesh Kumar GARG, Gurgaon (IN); Ajay Kumar SINGLA, Gurgaon (IN)

(21) Appl. No.: 15/321,074

(22) PCT Filed: Jun. 23, 2015
(86) PCT No.: PCT/IB15/54707

§ 371 Date: Dec. 21, 2016

(30) Foreign Application Priority Data
Jun. 23, 2014 (IN) 1669/DEL/2014

(51) Int. Cl. A61K 31/519; A61K 9/20; B29C 43/00; B29B 7/88; B29B 13/10; A61K 9/28; A61J 3/10

(52) U.S. Cl. CPC A61K 31/519; A61K 9/284; A61K 9/2095; A61J 3/10; A61K 9/2018; A61K 9/2054; A61K 9/2013; B29B 7/88; B29B 13/10; B29C 43/003; B29K 2105/0035

(57) ABSTRACT

The present invention relates to a dry process for the preparation of pharmaceutical compositions of tofacitinib comprising tofacitinib and one or more pharmaceutically acceptable excipients. Dry processes, such as direct compression or dry granulation, involve fewer and simpler process steps, thus preventing the loss of the active ingredient during processing.

### FIELD OF THE INVENTION

The present invention relates to a dry process for the preparation of pharmaceutical compositions of tofacitinib comprising tofacitinib and one or more pharmaceutically acceptable excipients.

### BACKGROUND OF THE INVENTION

Tofacitinib citrate is a Janus kinase inhibitor, which is chemically designated as (3R,4R)-4-methyl-3-(methyl-7H-pyrrolo[2,3-d]pyrimidin-4-ylamino)-β-oxo-1-piperidinepropanenitrile, 2-hydroxy-1,2,3-propanetricarboxylate (1:1).

Processes for the preparation of tofacitinib are disclosed in U.S. Pat. Nos. RE41,783 and 7,301,023.

A process for the preparation of tofacitinib citrate is disclosed in U.S. Pat. No. 6,965,027.

U.S. Pat. No. 6,956,041 provides various dosage forms of tofacitinib for oral, parenteral, buccal, or intranasal administration. It further mentions pharmaceutical compositions for oral administration, for example, tablets or capsules prepared by conventional means with pharmaceutically acceptable excipients.

The above mentioned prior art references show that tofacitinib might be formulated into different possible immediate release dosage forms; however, no specific formulation has been disclosed.

There remains a need to develop alternate compositions of tofacitinib which have a simpler composition and simpler process steps.

### SUMMARY OF THE INVENTION

The present invention relates to a dry process for the preparation of pharmaceutical compositions of tofacitinib comprising tofacitinib and one or more pharmaceutically acceptable excipients.

Dry processes, such as direct compression or dry granulation, involve fewer and simpler process steps, thus preventing the loss of the active ingredient during processing. Further, the manufacturing time and the amount of equipment needed are also reduced significantly, which makes the process more cost effective than those in the prior art.

### DETAILED DESCRIPTION OF THE INVENTION

A first aspect of the present invention provides a dry process for the preparation of a pharmaceutical composition of tofacitinib, wherein the composition comprises tofacitinib or a pharmaceutical salt thereof, and one or more diluents, disintegrants, and pharmaceutically acceptable excipients; wherein the dry process is selected from direct compression and dry granulation.

According to one embodiment of this aspect, the pharmaceutically acceptable excipients are selected from the group comprising binders, lubricants, glidants, and coating additives.

A second aspect of the present invention provides a dry process for the preparation of a pharmaceutical composition of tofacitinib comprising:
(i) blending tofacitinib and a diluent;
(ii) blending the blend of step (i) with a disintegrant and a diluent;
(iii) blending a lubricant with the blend of step (ii); and
(iv) compressing the blend of step (iii) into tablets or filling into capsules.

A third aspect of the present invention provides a dry process for the preparation of a pharmaceutical composition of tofacitinib comprising:
(i) blending tofacitinib and a diluent;
(ii) blending the blend of step (i) with a disintegrant, a diluent, and a lubricant;
(iii) passing the blend of step (ii) through a roller compactor;
(iv) milling the material obtained from step (iii);
(v) blending a lubricant with the milled material of step (iv); and
(vi) compressing the blend of step (v) into tablets or filling into capsules.

The term “pharmaceutical composition” as used herein may include tablets, capsules, granules, and the like.

The term “tofacitinib” as used herein refers to tofacitinib free base or pharmaceutically acceptable salts, in particular pharmaceutically acceptable acid addition salts, e.g. citrate, hydrochloride, hydrobromide, hydroiodide, nitrate, sulfate, bisulfate, phosphate, acetate, lactate, acid citrate, tartarate, succinate, malate, maleate, oxalate, fumarate, gluconate, saccharate, benzoate, methansulfonate, ethanesulfonate, benzenesulfonate, and the like. In particular, the term “tofacitinib” refers to tofacitinib citrate.

Suitable diluents are selected from the group comprising lactose, e.g., lactose monohydrate, atomized lactose, lactose anhydrous, spray dried lactose, and agglomerated lactose; microcrystalline cellulose, e.g., microcrystalline PH 112, microcrystalline PH 101, and microcrystalline PH 102; starch, e.g., pregelatinized starch and hydroxypropyl cellulose; ethyl cellulose; sugars, e.g., sucrose, Di-Pac® (a directly compressible, co-crystallized sugar consisting of 97% sucrose and 3% maltodextrin), maltrin, maltodextrin, mannitol, and maltose; dibasic calcium phosphate; and combinations thereof. In particular, the diluents are lactose monohydrate, microcrystalline cellulose, pregelatinized starch, or combinations thereof.

Suitable disintegrants are selected from the group comprising croscarmellose sodium, hydroxypropyl cellulose (L-HPC), crospovidone, carboxymethyl cellulose sodium, carboxymethyl cellulose calcium, sodium starch glycolate, gums, alginic acid or alginates, pregelatinized starch, corn starch, modified starch, carboxymethyl starch, polyacrylates, and combinations thereof. In particular, the disintegrant is croscarmellose sodium.

The term “pharmaceutically acceptable excipients” as used herein includes any physiologically inert additives that are routinely used in pharmaceutical dosage forms. Pharmaceutically acceptable excipients are selected from the group comprising binders, lubricants, glidants, coating additives or combinations thereof.

Suitable coating additives are selected from film-forming polymers, plasticizers, anti-foaming agents, opacifiers, anti-tacking agents, coloring agents, coating solvents, and combinations thereof.

Suitable binders are selected from the group comprising starch, e.g., pregelatinized starch and low density starch; povidone; copovidone; microcrystalline cellulose; lactose; cellulose, e.g., hydroxypropylmethyl cellulose, hydroxypropyl cellulose, hydroxyethyl cellulose, methyl cellulose, and ethyl cellulose; xanthan gum; gum acacia; gum arabic; tragacanth; sorbitol; dextrose; sucrose; mannitol; gelatin; pullulan; sodium alginate; propylene glycol; polyvinyl alcohol; corn syrup; methacrylates; carboxyvinyl polymers like carbomers; and combinations thereof.

Suitable glidants are selected from the group comprising magnesium stearate, stearic acid, calcium stearate, Aerosil® (colloidal silicon dioxide), starch, talc, and combinations thereof.

Suitable lubricants are selected from the group comprising common minerals like magnesium stearate, talc, and silica; fats, e.g., vegetable stearin and hydrogenated castor oil; sucrose esters of fatty acid; sodium stearyl fumarate; wax, e.g., microcrystalline wax, yellow beeswax, and white beeswax; and combinations thereof.

Suitable film-forming polymers are selected from the group comprising hydroxypropylmethyl cellulose, ethyl cellulose, methyl cellulose, hydroxyethyl cellulose, hydroxypropyl cellulose, sodium carboxymethyl cellulose, cellulose acetate, polyvinylpyrrolidone, polyvinylalcohol, polyethylene glycol, and combinations thereof. A preferred film-forming polymer is hydroxypropylmethyl cellulose. Other suitable film-forming polymers which are known in the art may also be used. Many suitable film coating products are commercially available e.g., Opadry® and Opaglos®.

Suitable plasticizers are selected from the group comprising triethyl citrate, dibutyl sebacate, acetylated triacetin, tributyl citrate, glyceryl tributyrate, monoglyceride, rapeseed oil, olive oil, sesame oil, acetyl tributyl citrate, acetyl triethyl citrate, glycerin, sorbitol, diethyl oxalate, diethyl phthalate, diethyl malate, diethyl fumarate, dibutyl succinate, diethyl malonate, dioctyl phthalate, and combinations thereof.

A suitable anti-foaming agent is simethicone. Simethicone imparts smoothness to coating.

Suitable opacifiers are selected from the group comprising titanium dioxide, manganese dioxide, iron oxide, silicon dioxide, and combinations thereof.

Suitable anti-tacking agents are selected from the group comprising talc, magnesium stearate, calcium stearate, stearic acid, silica, glyceryl monostearate, and combinations thereof.

Suitable coloring agents are selected from the group consisting of FD&C (Federal Food, Drug and Cosmetic Act) approved coloring agents; natural coloring agents; natural juice concentrates; pigments, e.g. iron oxide, titanium dioxide, and zinc oxide; and combinations thereof.

Suitable coating solvents used for forming a solution or suspension for coating are selected from the group comprising water, ethanol, methylene chloride, isopropyl alcohol, acetone, methanol, and combinations thereof.

The term “dry process” as used herein may include direct compression or dry granulation.

“Direct compression” refers to a process which involves blending the ingredients and then compressing into tablets.

“Dry granulation” refers to a process which involves blending the ingredients followed by compaction and size reduction of the mix in order to produce a granular blend of uniform size. The granules so obtained may be compressed into tablets or filled into capsules of a suitable size.

Coating may be performed by applying the coating composition as a solution, suspension, or blend using any conventional coating technique known in the art, such as spray coating in a conventional coating pan, fluidized bed processors, dip coating, or compression coating.

The invention is further illustrated by the following examples, which are for illustrative purposes only and should not be construed as limiting the scope of the invention in any way.

### EXAMPLES

### Example 1

| | | |
|---|---|---|
| S. No. | Ingredients | Quantity per tablet (mg) |

Core

| | | |
|---|---|---|
| 1 | Tofacitinib citrate | 8.00 |
| 2 | Lactose monohydrate | 90.00 |
| 3 | Microcrystalline cellulose | 90.00 |
| 4 | Croscarmellose sodium | 11.00 |
| 5 | Magnesium stearate | 1.00 |
| | Core Tablet Weight | 200.00 |

Film Coating

| | | |
|---|---|---|
| 6 | Opadry ® white | 6.00 |
| 7 | Purified water | q.s. |
| | Coated Tablet Weight | 206.00 |

### Procedure:

1. Tofacitinib citrate and half the quantity of lactose monohydrate were sifted and blended.
2. Microcrystalline cellulose, croscarmellose sodium, and the remaining quantity of lactose monohydrate were sifted.
3. The blend of step 1 was blended with the sifted material of step 2.
4. Magnesium stearate was sifted, and then blended with the blend of step 3.
5. The blend of step 4 was compressed to obtain tablets using suitable tooling.
6. Opadry® white was dispersed in purified water.
7. The tablets of step 5 were coated with the dispersion of step 6.

### Example 2

| | | |
|---|---|---|
| S. No. | Ingredients | Quantity per tablet (mg) |

Core

| | | |
|---|---|---|
| 1 | Tofacitinib citrate | 8.00 |
| 2 | Pregelatinized starch | 40.00 |
| 3 | Microcrystalline cellulose | 140.00 |
| 4 | Croscarmellose sodium | 11.00 |
| 5 | Magnesium stearate | 1.00 |
| | Core Tablet Weight | 200.00 |

Film Coating

| | | |
|---|---|---|
| 6 | Opadry ® white | 6.00 |
| 7 | Purified water | q.s. |
| | Coated Tablet Weight | 206.00 |

### Procedure:

1. Tofacitinib citrate, pregelatinized starch, and croscarmellose sodium were sifted and blended.
2. Microcrystalline cellulose was sifted.
3. The blend of step 1 was blended with the sifted material of step 2.
4. Magnesium stearate was sifted, and then blended with the blend of step 3.
5. The blend of step 4 was compressed to obtain tablets using suitable tooling.
6. Opadry® white was dispersed in purified water.
7. The tablets of step 5 were coated with the dispersion of step 6.

### Example 3

| | | |
|---|---|---|
| S. No. | Ingredients | Quantity per tablet (mg) |

Core

| | | |
|---|---|---|
| 1 | Tofacitinib citrate | 8.00 |
| 2 | Lactose monohydrate | 90.00 |
| 3 | Microcrystalline cellulose | 90.00 |
| 4 | Croscarmellose sodium | 11.00 |
| 5 | Magnesium stearate | 1.00 |
| | Core Tablet Weight | 200.00 |

Film Coating

| | | |
|---|---|---|
| 6 | Opadry ® white | 6.00 |
| 7 | Purified water | q.s. |
| | Coated Tablet Weight | 206.00 |

### Procedure:

1. Tofacitinib citrate and half the quantity of lactose monohydrate were sifted and blended.
2. Microcrystalline cellulose, croscarmellose sodium, the remaining quantity of lactose monohydrate, and half the quantity of magnesium stearate were sifted.
3. The blend of step 1 was blended with the sifted material of step 2.
4. The remaining quantity of magnesium stearate was sifted, and then blended with the blend of step 3.
5. The blend of step 4 was passed through a roller compactor.
6. The compacted material of step 5 was milled.
7. The milled material of step 6 was compressed to obtain tablets using suitable tooling.
8. Opadry® white was dispersed in purified water.
9. The tablets of step 7 were coated with the dispersion of step 8.

We claim:

1. A dry process for the preparation of a pharmaceutical composition of tofacitinib, wherein the composition comprises tofacitinib or a pharmaceutical salt thereof, and one or more diluents, disintegrants, and pharmaceutically acceptable excipients; wherein the dry process is selected from direct compression and dry granulation.

2. The dry process according to claim 1, wherein the pharmaceutically acceptable excipients are selected from the group comprising binders, lubricants, glidants, and coating additives.

3. A dry process for the preparation of a pharmaceutical composition of tofacitinib comprising:
i. blending tofacitinib and a diluent;
ii. blending the blend of step (i) with a disintegrant and a diluent;
iii. blending a lubricant with the blend of step (ii); and
iv. compressing the blend of step (iii) into tablets or filling into capsules.

4. A dry process for the preparation of a pharmaceutical composition of tofacitinib comprising the steps of:
i. blending tofacitinib and a diluent;
ii. blending the blend of step (i) with a disintegrant, a diluent, and a lubricant;
iii. passing the blend of step (ii) through a roller compactor;
iv. milling the material obtained from step (iii);
v. blending a lubricant with the milled material of step (iv); and
vi. compressing the blend of step (v) into tablets or filling into capsules.
